(19) United States
(12) Patent Application Publication
(10) Pub. No.: US 2005/0085461 A1
(43) Pub. Date: Apr. 21, 2005

(54) Benzenesulfonamide derivatives

(76) Inventors: David G Cooper, Essex (GB); Ian T Forbes, Essex (GB); Andrew D Gribble, Essex (GB); Andrew P Lightfoot, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/504,110

(22) PCT Filed: Feb. 13, 2003
(86) PCT No.: PCT/EP03/01544

(30) Foreign Application Priority Data
Feb. 13, 2002 (GB) 0203435.3
Feb. 28, 2002 (GB) 0204721.5

(51) Int. Cl. A61K 31/55; A61K 31/47; C07D 217/22; C07D 223/16

(52) U.S. Cl. 514/217.01; 514/310; 540/594; 546/143

(57) ABSTRACT

The invention provides compounds of formula (I) 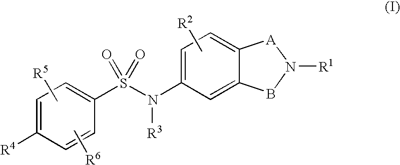 wherein
A and B represent the groups CH₂)ₘ— and CH₂)ₙ— respectively;
R¹ represents hydrogen or C₁₋₄alkyl;
R² represents hydrogen, halogen, hydroxy, cyano, nitro, hydroxyC₁₋₆alkyl, trifluoromethyl, trifluoromethoxy, C₁₋₆alkyl, C₁₋₆alkoxy, —(CH₂)ₚC₃₋₆cycloalkyl, —(CH₂)ₚC₃₋₆cycloalkyloxy, —COC₁₋₆alkyl, —SO₂C₁₋₆alkyl, —SOC₁₋₆alkyl, —S-C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl or a fused bicyclic heterocyclic ring system;
R³ represents hydrogen or C₁₋₆alkyl;
R⁴ represents halogen, trifluoromethyl, trifluoromethoxy, C₁₋₆alkyl, C₁₋₆alkoxy, —(CH₂)ₚC₃₋₆cycloalkyl or —(CH₂)ₚC₃₋₆cycloalkyloxy;
R⁵ and R⁶ each independently represent hydrogen, halogen, hydroxy, cyano, nitro, hydroxyC₁₋₆alkyl, trifluoromethyl, trifluoromethoxy, C₁₋₆alkyl, C₁₋₆alkoxy, —(CH₂)ₚC₃₋₆cycloalkyl, —(CH₂)ₚC₃₋₆cycloalkyloxy, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl or a fused bicyclic heterocyclic ring system;
R⁷ and R⁸ each independently represent hydrogen or C₁₋₆alkyl; m and n independently represent an integer selected from 1 and 2; p independently represents an integer selected from 0, 1, 2 and 3; or a pharmaceutically acceptable salt or solvate thereof, with the proviso that the compounds 4-methyl-N-(1,2,3,4-tetrahydroisoquinolin-6-yl)-benzenesulfonamide, 7-(4-chlorophenyl)sulfonamido-1,2,3,4-tetrahydroisoquinoline hydrochloride and N-(2-ethyl-5-isoindolinyl)-p-toluenesulfonamide are excluded. The compounds are useful in therapy, in particular as antipsychotic agents.

This invention relates to novel compounds, pharmaceutical compositions containing them and their use in therapy, in particular as antipsychotic agents.

EP266949 describes tetrahydroisoquinolin-2-yl derivatives of carboxylic acids as thromboxane A2 antagonists.

U.S. Pat. No. 4,321,254 describes antiallergic imidodisulfamides. 7-(4-Chlorophenylsulphonamido)-1,2,3,4-tetrahydroisoquinoline hydrochloride is disclosed as an intermediate in the preparation of imidodisulfamides.

WO96/35713 and WO96/38471 describe dipeptides which promote the release of growth hormone. 4-Methyl-(N-(N-(1,2,3,4-tetrahydroisoquinolin-6-yl)-benzenesulfonamide is disclosed as an intermediate in the preparation of these peptides in both of these applications.

N-(2-Ethyl-5-isoindolinyl)-p-toluenesulfonamide is cited in Beilstein (CAS Registry Number 3606-744) as being disclosed in patent DE36431. However, this citation is apparently erroneous, as no disclosure of this compound is made in DE36431.

WO 01/62737 discloses amino pyrazole derivatives useful for the treatment of obesity and other disorders associated with the NPY receptor subtype Y5.

EP0937723 discloses sulfonamide compounds useful in the treatment of thrombolytic disorders.

WO 01/85695 discloses tetrahydroisoquinoline analogues useful as growth hormone secretagogues.

U.S. Pat. No. 5,684,195 discloses a method of preparing sulfonamides from sulfones.

WO 02/46164 discloses aryl sulfonamide compounds that are said to be useful as selective

ER-β ligands in the treatment or prophylaxis of Alzheimer's disease, anxiety disorders, depressive disorders, osteoporosis, cardiovascular disease, rheumatoid arthritis or prostate cancer.

According to the invention, there is provided a compound of formula (I): 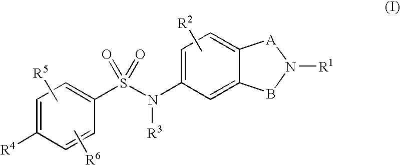
wherein
A and B represent the groups CH₂)ₘ— and CH₂)ₙ— respectively;
R¹ represents hydrogen or C₁₋₆alkyl;
R² represents hydrogen, halogen, hydroxy, cyano, nitro, hydroxyC₁₋₆alkyl, trifluoromethyl, trifluoromethoxy, C₁₋₆alkyl, C₁₋₆alkoxy, —(CH₂)ₚC₃₋₆cycloalkyl, —(CH₂)ₚC₃₋₆cycloalkyloxy, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl or a fused bicyclic heterocyclic ring system;
R³ represents hydrogen or C₁₋₆alkyl;
R⁴ represents halogen, trifluoromethyl, trifluoromethoxy, C₁₋₆alkyl, C₁₋₆alkoxy, —(CH₂)ₚC₃₋₆cycloalkyl or —(CH₂)ₚC₃₋₆cycloalkyloxy;
R⁵ and R⁶ each independently represent hydrogen, halogen, hydroxy, cyano, nitro, hydroxyC₁₋₆alkyl, trifluoromethyl, trifluoromethoxy, C₁₋₆alkyl, C₁₋₆alkoxy, CH₂)C₃₋₆cycloalklyl, —(CH₂)ₚC₃₋₆cycloalxyloxy, —COC₁₋₆alkyl, —SO₂C₁₋₆alkyl, SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl or a fused bicyclic heterocyclic ring system;
R⁷ and R⁸ each independently represent hydrogen or C₁₋₆alkyl;
m and n independently represent an integer selected from 1 and 2;
p independently represents an integer selected from 0, 1, 2 and 3;
or a pharmaceutically acceptable salt or solvate thereof,
   with the proviso that the compounds 4-methyl-N-(1,2,3,4-tetrahydroisoquinolin-6-yl)-benzenesulfonamide, 7-(4-chlorophenyl)sulfonamido-1,2,3,4-tetrahydroisoquinoline hydrochloride and N-(2-ethyl-5-isoindolinyl)-p-toluenesulfonamide are excluded.

As a further aspect of the invention, there is provided a compound of formula (I) or a pharmaceutically acceptable salt or solvate thereof, wherein groups A, B and R¹ to R⁶ have any of the meanings as given hereinbefore, with the proviso that when R¹ and R³ both represent hydrogen and A and B both represent (CH₂)₂, R⁴ does not represent methyl or ethyl.

As a further aspect of the invention, there is provided a compound of formula (I) or a pharmaceutically acceptable salt or solvate thereof, wherein groups A, B and R¹ to R⁶ have any of the meanings as given hereinbefore, with the proviso that when R¹, R² and R³ all represent hydrogen and A and B both represent (CH₂)₂, R⁴ does not represent methyl or ethyl.

As a further aspect of the invention, there is provided a compound of formula (I) or a pharmaceutically acceptable salt or solvate thereof, wherein groups A, B and R¹ to R⁶ have any of the meanings as given hereinbefore, with the proviso that when A and B both represent (CH₂)₂, R³ represents hydrogen and R⁴ represents halogen, R¹ does not represent hydrogen.

As a further aspect of the invention, there is provided a compound of formula (I) or a pharmaceutically acceptable salt or solvate thereof, wherein groups A, B and R¹ to R⁶ have any of the meanings as given hereinbefore, with the proviso that when A and B both represent (CH₂)₂, R² and R³ both represent hydrogen and R⁴ represents halogen, R¹ does not represent hydrogen.

As used herein, the term “alkyl” refers to straight or branched hydrocarbon chains containing the specified number of carbon atoms. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, n-hexyl, isobutyl, isopropyl, t-butyl and 1,1-dimethylpropyl.

As used herein, the term “alkoxy” refers to a straight or branched alkoxy group containing the specified number of carbon atoms. For example, C₁₋₆alkoxy means a straight or branched alkoxy group containing at least 1, and at most 6, carbon atoms. Examples of “alkoxy” as used herein include, but are not limited to, methoxy, ethoxy, propoxy, prop-2-oxy, n-butoxy, but-2-oxy, 2-methylprop-1-oxy, 2-methylprop-2-oxy, pentoxy or hexyloxy.

As used herein, the term “cycloalkyl” refers to a non-aromatic hydrocarbon ring containing the specified number of carbon atoms. For example, C₃₋₇cycloalkyl means a non-aromatic ring containing at least three, and at most seven, ring carbon atoms. Examples of “cycloalkyl” as used herein include, but are not limited to, cyclopropyl, cyclobutyl, cyclopentyl, cyclohexyl and cycloheptyl. A C₆₋₇cycloalkyl group is preferred.

As used herein, the term “halogen” refers to the elements fluorine, chlorine, bromine and iodine.

As used herein, the term “aryl” refers to a phenyl ring or a naphthyl ring. As used herein, the term “heteroaryl” refers to a 5- or 6-membered heterocyclic aromatic ring or a fused bicyclic heterocyclic ring system.

As used herein, the term “5- or 6-membered heterocyclic aromatic ring” refers to a monocyclic unsaturated ring containing at least one heteroatom independently selected from oxygen, nitrogen and sulfur. Examples of suitable 5- and 6-membered heterocyclic aromatic rings include, but are not limited to, furyl, thienyl, pyrrolyl, oxazolyl, thiazolyl, imidazolyl, oxadiazolyl, thiadiazolyl, pyridyl, triazolyl, triazinyl, pyridazyl, pyrimidinyl, pyrazolyl, isothiazolyl and isoxazolyl.

As used herein, the term “fused bicyclic heterocyclic ring system” refers to a ring system comprising two 5- to 7-membered saturated or unsaturated rings, the ring system containing at least one heteroatom independently selected from oxygen, nitrogen and sulfur. Preferably, each ring has 5 or 6 ring atoms. Examples of suitable fused bicyclic rings include, but are not limited to, indolyl, indolinyl, benzofuranyl, benzothiophenyl, quinolyl, isoquinolyl, tetrahydroquinolyl, benzodioxanyl, indanyl and tetrahydronapthyl. Further examples include, but are not limited to, quinolizinyl, naphthyridinyl, quinoxalinyl, quinazolinyl, cinnolinyl, isoindolyl, indolizinyl, indazolyl, pyrrolopyridinyl, benzimidazolyl, benzoxazolyl, benzisoxazolyl, benzothiazolyl, benzisothiazolyl, benzoxadiazolyl, dihydrobenzothienyl, dihydrobenzofuranyl, benzodioxolanyl, methylenedioxyphenyl, dihydrobenzodioxinyl and the like.

As used herein, the term “substituted” refers to substitution with the named substituent or substituents, multiple degrees of substitution being allowed unless otherwise stated. As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include water, methanol, ethanol and acetic acid. Most preferably the solvent used is water and the solvate may also be referred to as a hydrate.

It will be appreciated that for use in medicine the salts of formula (I) should be physiologically acceptable. Suitable physiologically acceptable salts will be apparent to those skilled in the art and include for example acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Other non-physiologically acceptable salts e.g. oxalates, may be used, for example in the isolation of compounds of formula (I) and are included within the scope of this invention. Also included within the scope of the invention are solvates and hydrates of the compounds of formula (I).

Certain of the compounds of formula (I) may form acid addition salts with one or more equivalents of the acid. The present invention includes within its scope all possible stoichiometric and non-stoichiometric forms thereof.

Certain compounds of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. Likewise, it is understood that compounds of formula (I) may exist in tautomeric forms other than that shown in the formula and these are also included within the scope of the present invention.

The groups R², R⁵ and R⁶ may be located on any position on their respective phenyl rings.

When R², R¹ or R⁶ represent optionally substituted aryl or optionally substituted heteroaryl, the optional substituents may be independently selected from C₁₋₆alkyl, C₁₋₆alkoxy, halogen, trifluoromethyl, trifluoromethoxy, cyano and —S—C₁₋₆alkyl.

Preferably, R¹ represents hydrogen or C₁₋₄alkyl. More preferably, R¹ represents hydrogen, methyl, ethyl, n-propyl or isopropyl. Even more preferably, R¹ represents hydrogen, methyl or isopropyl.

In a more preferred embodiment, the R² group is located at the para-position relative to the group B.

Preferably, R² represents hydrogen, halogen, C₁₋₆alkyl or C₁₋₆alkoxy. More preferably, R² represents hydrogen, halogen, C₁₋₄alkyl or C₁₋₄alkoxy. Even more preferably, R² represents hydrogen, bromine, ethyl, methoxy, ethoxy or isopropoxy. Even more preferably, R² represents hydrogen, halogen, C₁₋₄alkyl or C₁₋₄alkoxy located at the para-position relative to the group B.

i.e. a compound of formula (IA) 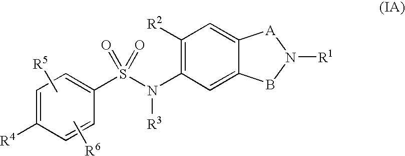
or a pharmaceutically acceptable salt or solvate thereof wherein groups A, B and R¹ to R⁶ have any of the meanings as given hereinbefore.

For compounds of the formula (I) or (IA), preferably, R³ represents hydrogen or C₁₋₄alkyl. More preferably, R³ represents hydrogen, methyl, ethyl, n-propyl or isopropyl. Even more preferably, R³ represents hydrogen, methyl or isopropyl.

For compounds of the formula (I) or (IA), preferably, R⁴ represents C₁₋₆alkyl, C₁₋₆alkoxy, C₃₋₆cycloalkyl, halogen, trifluoromethyl or trifluoromethoxy. More preferably, R⁴ represents C₁₋₆alkyl, C₁₋₄alkoxy, iodine, cyclohexyl, trifluoromethyl or trifluoromethoxy.

For compounds of the formula (I) or (IA), preferably, the optional substituents for the groups R², R⁵ and R⁶ are selected from chlorine, fluorine, bromine, methyl, ethyl, t-butyl, methoxy, trmfluoromethyl, trifluoromethoxy, cyano and 4-methyl.

For compounds of the formula (I) or (IA), preferably, R⁵ and R⁶ independently represent hydrogen.

For compounds of the formula (I) or (L), preferably, R⁷ and R⁸ independently represent hydrogen or C₁₋₄alkyl. More preferably, R⁷ and R⁸ independently represent hydrogen or methyl.

For compounds of the formula (I) or (IA), preferably, p represents 0.

In a preferred aspect, m is 1 and n is 1 and the invention is a compound of formula (IB): 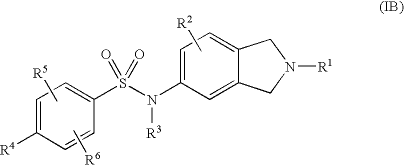
or a pharmaceutically acceptable salt or solvate thereof wherein groups R¹ to R⁶ have any of the meanings as given hereinbefore.

In a preferred aspect, m is 2 and n is 1 and the invention is a compound of formula (IC): 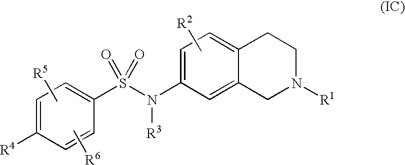
or a pharmaceutically acceptable salt or solvate thereof wherein groups R¹ to R⁶ have any of the meanings as given hereinbefore.

In a preferred aspect, m is 1 and n is 2 and the invention is a compound of formula (ID): 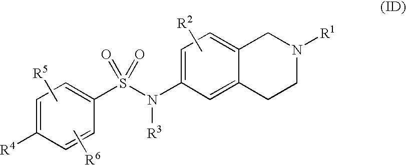
or a pharmaceutically acceptable salt or solvate thereof wherein groups R¹ to R⁶ have any of the meanings as given hereinbefore.

In a preferred aspect, m is 2 and n is 2 and the invention is a compound of formula (E): 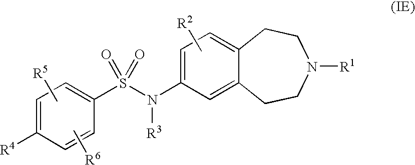
or a pharmaceutically acceptable salt or solvate thereof wherein groups R¹ to R⁶ have any of the meanings as given hereinbefore.

In a preferred aspect, m is 2 and n is 2 and R² is located at the para-position relative to the group B i.e. the invention is a compound of formula (IF): 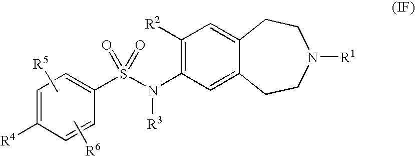
or a pharmaceutically acceptable salt or solvate thereof wherein groups R¹ to R⁶ have any of the meanings as given hereinbefore.

According to a further aspect of the invention, there is provided a compound of formula (I) or a pharmaceutically acceptable salt or solvate thereof wherein the groups A, B and R¹ to R⁶ have any of the meanings as given hereinbefore and Z represents oxygen or C₁₋₆ alkylene.

According to a further aspect of the invention, there is provided a compound of formula (A) or a pharmaceutically acceptable salt or solvate thereof wherein the groups A, B and R¹ to R⁶ have any of the meanings as given hereinbefore and Z represents oxygen or C₁₋₆ alkylene.

According to a further aspect of the invention, there is provided a compound of formula (IB) or a pharmaceutically acceptable salt or solvate thereof wherein the groups R¹ to R⁶ have any of the meanings as given hereinbefore and Z represents oxygen or C₁₋₆ alkylene.

According to a further aspect of the invention, there is provided a compound of formula (IC) or a pharmaceutically acceptable salt or solvate thereof wherein the groups R¹ to R⁶ have any of the meanings as given hereinbefore and Z represents oxygen or C₁₋₆ alkylene.

According to a further aspect of the invention, there is provided a compound of formula (ID) or a pharmaceutically acceptable salt or solvate thereof wherein the groups R¹ to R⁶ have any of the meanings as given hereinbefore and Z represents oxygen or C₁₋₆ alkylene.

According to a further aspect of the invention, there is provided a compound of formula (IE) or a pharmaceutically acceptable salt or solvate thereof wherein the groups R¹ to R⁶ have any of the meanings as given hereinbefore and Z represents oxygen or C₁₋₆ alkylene.

According to a further aspect of the invention, there is provided a compound of formula (IF) or a pharmaceutically acceptable salt or solvate thereof wherein the groups R¹ to R⁶ have any of the meanings as given hereinbefore and Z represents oxygen or C₁₋₆ alkylene.

In a preferred aspect compounds of formula (I) are of the formula (IB), (IC), (IE) and (IF) or a pharmaceutically acceptable salt or solvate thereof wherein groups R¹ to R⁶ have any of the meanings as given hereinbefore.

Particular compounds according to the invention include those incorporated in Tables 1 to 3 and those specifically exemplified and named hereinafter including, without limitation:—
4-Butyl-N-(2,3,4,5-tetrahydro-1H-3-benzazepine-7-yl)-benzenesulfonamide;
4-Butyl-N-(3-methyl-2,3,4,5-tetrahydro-1H-3-benzazepine-7-yl)-benzenesulfonamide;
4-Butyl-N-methyl-N-(2,3,4,5-tetrahydro-1H-3-benzazepine-7-yl)-benzenesulfonamide hydrochloride;
4-Butyl-N-methyl-N-(3-methyl-2,3,4,5-tetrahydro-1H-3-benzazepine-7-yl)-benzenesulfonamide hydrochloride;
4-Butyl-N-(8-methoxy-2,3,4,5-tetrahydro-1H-3-benzazepine-7-yl)-benzenesulfonamide hydrochloride;
4-Butyl-N-(3-methyl-8-methoxy-2,3,4,5-tetrahydro-1H-3-benzazepine-7-yl)-benzenesulfonamide hydrochloride;
4-Butyl-N-(1,2,3,4-tetrahydro-isoquinolin-7-yl)-benzenesulfonamide;
4-Butyl-N-(2,3-dihydro-1H-isoindol-5-yl)-benzenesulfonamide hydrochloride; and
4-Butyl-N-(2-methyl-2,3-dihydro-1H-isoindol-5-yl)-benzenesulfonamide.

The compounds of the present invention may be in the form of their free base or physiologically acceptable salts thereof, particularly the monohydrochloride or monomesylate salts.

The present invention also provides a general process (A) for preparing compounds of formula (I) which process comprises:
reacting a compound of formula (II) 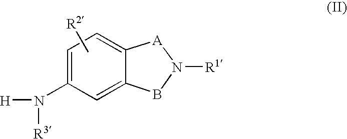
with a compound of formula (III) 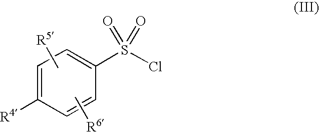
wherein R^{1′}-R^{6′} represent R¹ to R⁶ as hereinbefore defined, or are groups that may be readily convertible to R¹ to R⁶. For example, conversion of an R^{4′} bromo substituent to an R⁴ alkyl group can be achieved by Kumada coupling i.e. treatment of the bromo compound with an alkyl Grignard reagent in the presence of a palladium catalyst.

This general method (A) can be conveniently performed by mixing the two components in a suitable solvent such as pyridine or dichloromethane (in the presence of a base), at 0° C.

The present invention also provides a general process (B) for preparing compounds of formula (I) which process comprises:
converting a compound of formula (I) 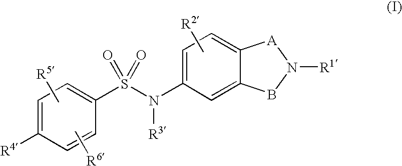
wherein the substituents R^{1′} to R^{5′} are the same as in formula (I) or convertible into another compound of formula (I) (using conventional techniques).

Interconversion of one of the R^{1′} to R^{5′} groups to the corresponding R¹ to R⁵ group typically arises when one compound of formula (I) is used as the immediate precursor of another compound of formula (I), or when it is easier to introduce a more complex or reactive substituent at the end of a synthetic sequence.

For example, conversion of R^{1′} from a BOC group to hydrogen is conducted by the treatment of the N-BOC protected compound with hydrogen chloride in ethanol or dioxan at room temperature.

Conversion of R^{1′} from hydrogen to an alkyl group is conducted by the treatment of the NH compound with the appropriate aldehyde in dichloroethane in the presence of a reducing agent, such as sodium triacetoxyborohydride, or by the treatment of the NH compound with the appropriate alkyl halide, such as iodomethane, under standard alkylation conditions (potassium carbonate in DMF at 60° C.).

Conversion of R^{3′} from hydrogen to an alkyl group is conducted by the treatment of the sulfonamide NH compound with the appropriate alcohol, such as methanol, under Mitsunobu conditions i.e. treatment with diisopropyl azodicarboxylate/triphenylphosphine and methanol in tetrahydrofuran at room temperature.

Compounds of formula (II) are known in the literature or can be prepared by known processes, for example the reduction of the corresponding nitro compound by catalytic hydrogenation as described in WO99/14197. Suitable examples of an R^{1′} protecting group are trifluoroacetyl or the t-butoxycarbonyl (BOC) group.

Compounds of formula (III) are commercially available or may be prepared by established procedures, for example chlorosulfonylation of a suitable substituted aromatic precursor, using chlorosulfonic acid, for example as described in Bull. Soc. Chim. France, 1964, (2), 248-250.

Compounds of formula (I) have been found to exhibit affinity for dopamine receptors, in particular the D₃ and D₂ receptors, and are useful in the treatment of disease states which require modulation of such receptors, such as psychotic conditions. Many of the compounds of formula (I) have also been found to have greater affinity for dopamine D₃ than for D₂ receptors. The therapeutic effect of many antipsychotic agents (neuroleptics) is generally believed to be exerted via blockade of D₂ receptors; however this mechanism is also thought to be responsible for undesirable extrapyramidal side effects (eps) associated with many neuroleptic agents. Without wishing to be bound by theory, it has been suggested that blockade of the dopamine D₃ receptor may give rise to beneficial antipsychotic activity without significant eps. (see for example Sokoloff et al, Nature, 1990; 347: 146-151; and Schwartz et al, Clinical Neuropharmacology, Vol 16, No. 4, 295-314, 1993). Additionally, certain compounds of formula (I) have antagonist affinity for the serotonin 5-HT_{2A}, 5-HT_{2C} and 5-HT₆ receptors. These additional properties may give rise to enhanced anti-psychotic activity (e.g. improved effects on cognitive dysfunction) and/or reduced eps. These could include, but are not limited to, attenuation of cognitive symptoms via 5-HT₆ receptor blockade (see Reavill, C. and Rogers, D. C., 2001, Investigational Drugs 2, 104-109), and reduced anxiety (see for example Kennett et al., Neuropharmacology 1997 April-May; 36 (4-5): 609-20), protection against EPS (Reavill et al., Brit. J. Pharmacol., 1999; 126: 572-574) and antidepressant activity (Bristow et al., Neuropharmacology 39:2000; 1222-1236) via 5-HT_{2C} receptor blockade.

Compounds of formula (I) may also exhibit affinity for other receptors not mentioned above, resulting in beneficial antipyschotic activity.

The compounds of formula (I) are of use as antipsychotic agents for example in the treatment of schizophrenia, schizo-affective disorders, schizophreniform diseases, psychotic depression, mania, acute mania, paranoid and delusional disorders. Furthermore, they may have utility as adjunct therapy in Parkinsons Disease, particularly with compounds such as L-DOPA and possibly dopaminergic agonists, to reduce the side effects experienced with these treatments on long term use (e.g. see Schwartz et al., Brain Res. Reviews, 1998, 26, 236-242). From the localisation of D₃ receptors, it could also be envisaged that the compounds could also have utility for the treatment of substance abuse where it has been suggested that D3 receptors are involved (e.g. see Levant, 1997, Pharmacol. Rev., 49, 231-252). Examples of such substance abuse include alcohol, cocaine, heroin and nicotine abuse. Other conditions which may be treated by the compounds include dyskinetic disorders such as Parkinson's disease, neuroleptic-induced parkinsonism and tardive dyslinesias; depression; anxiety; agitation; tension; social or emotional withdrawal in psychotic patients; cognitive impairment including memory disorders such as Alzheimer's disease; psychotic states associated with neurodegenerative disorders, e.g. Alzheimer's disease; eating disorders; obesity; sexual dysfunction; sleep disorders; emesis; movement disorders; obsessive-compulsive disorders; amnesia; aggression; autism; vertigo; dementia; circadian rhythm disorders; and gastric motility disorders e.g. IBS.

Therefore, the invention provides a compound of formula (I) as hereinbefore described or a pharmaceutically acceptable salt or solvate thereof for use in therapy.

The invention also provides a compound of formula (I) as hereinbefore described or a pharmaceutically acceptable salt or solvate thereof for use in a condition which requires modulation of a dopamine receptor.

The invention also provides a compound of formula (I) as hereinbefore described or a pharmaceutically acceptable salt or solvate thereof for use in the treatment of psychotic disorders, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

The invention also provides the use of a compound of formula (I) as hereinbefore described or a pharmaceutically acceptable salt or solvate thereof in the manufacture of a medicament for the treatment of a condition which requires modulation of a dopamine receptor.

The invention also provides the use of a compound of formula (I) as hereinbefore described or a pharmaceutically acceptable salt or solvate thereof in the manufacture of a medicament for the treatment of psychotic disorders, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

The invention also provides a method of treating a condition which requires modulation of a dopamine receptor, which comprises administering to a mammal in need thereof an effective amount of a compound of formula (I) as hereinbefore described or a pharmaceutically acceptable salt or solvate thereof.

In a further aspect, the invention provides a method of treating psychotic disorders, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders which comprises administering to a mammal in need thereof an effective amount of a compound of formula (I) as hereinbefore described or a pharmaceutically acceptable salt or solvate thereof.

A preferred use for dopamine antagonists according to the present invention is in the treatment of psychotic disorders, Parkinsons disease, substance abuse, dyskinetic disorders, depression, bipolar disorder, anxiety, cognitive impairment.

“Treatment” includes prophylaxis, where this is appropriate for the relevant condition(s). For use in medicine, the compounds of the present invention are usually administered as a standard pharmaceutical composition. The present invention therefore provides in a further aspect a pharmaceutical composition comprising a compound of formula (I) as hereinbefore described or a pharmaceutically (i.e. physiologically) acceptable salt thereof and a pharmaceutically (i.e. physiologically) acceptable carrier. The pharmaceutical composition can be for use in the treatment of any of the conditions described herein.

The compounds of formula (I) as hereinbefore described may be administered by any convenient method, for example by oral, parenteral (e.g. intravenous), buccal, sublingual, nasal, rectal or transdermal administration and the pharmaceutical compositions adapted accordingly.

The compounds of formula (I) as hereinbefore described and their pharmaceutically acceptable salts which are active when given orally can be formulated as liquids or solids, for example syrups, suspensions or emulsions, tablets, capsules and lozenges.

A liquid formulation will generally consist of a suspension or solution of the compound or pharmaceutically acceptable salt in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active substance in a pharmaceutically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluorochlorohydrocarbon. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

Preferably the composition is in unit dose form such as a tablet, capsule or ampoule.

Each dosage unit for oral administration contains preferably from 1 to 250 mg (and for parenteral administration contains preferably from 0.1 to 25 mg) of a compound of the formula (I) or a pharmaceutically acceptable salt thereof calculated as the free base.

The pharmaceutically acceptable compounds of the invention will normally be administered in a daily dosage regimen (for an adult patient) of, for example, an oral dose of between 1 mg and 500 mg, preferably between 10 mg and 400 mg, e.g. between 10 and 250 mg or an intravenous, subcutaneous, or intramuscular dose of between 0.1 mg and 100 mg, preferably between 0.1 mg and 50 mg, e.g. between 1 and 25 mg of the compound of the formula (I) or a pharmaceutically acceptable salt thereof calculated as the free base, the compound being administered 1 to 4 times per day. Suitably the compounds will be administered for a period of continuous therapy, for example for a week or more.

Biological Test Methods

Binding Experiments on Cloned Dopamine (e.g. D2 and D3) Receptors

The ability of the compounds to bind selectively to human D2/D3 dopamine receptors can be demonstrated by measuring their binding to cloned receptors. The inhibition constants (Kᵢ) of test compounds for displacement of [¹²⁵I]-Iodosulpride binding to human D2/D3 receptors expressed in CHO cells were determined as follows. The cell lines were shown to be free from bacterial, fungal and mycoplasmal contaminants, and stocks of each were stored frozen in liquid nitrogen. Cultures were grown as monolayers or in suspension in standard cell culture media. Cells were recovered by scraping (from monolayers) or by centrifugation (from suspension cultures), and were washed two or three times by suspension in phosphate buffered saline followed by collection by centrifugation. Cell pellets were stored frozen at −80° C. Crude cell membranes were prepared by homogenisation followed by high-speed centrifugation, and characterisation of cloned receptors achieved by radioligand binding.

Preparation of CHO cell membranes: Cell pellets were gently thawed at room temperature, and resuspended in about 20 volumes of ice-cold Extraction buffer; 5 mM EDTA, 50 mM Trizma pre-set crystals (pH7.4@37° C.), 1 mM MgCl₂, 5 mM KCl and 120 mM NaCl. The suspension was homogenised using an Ultra-Turrax at full speed for 15 seconds. The homogenate was centrifuged at 18,000 r.p.m for 15 min at 4° C. in a Sorvall RC5C centrifuge. Supernatant was discarded, and homogenate re-suspended in extraction buffer then centrifugation was repeated. The final pellet was resuspended in 5 mM Trizma pre-set crystals (pH 7.4 @ 37° C.) and stored in 1 ml aliquot tubes at −80° C. (D2=3.0E+08 cells, D3=7.0E+07 cells and D4=1.0E+08 cells). The protein content was determined using a BCA protocol and bovine serum albumin as a standard (Smith, P. K., et al., Measurement of protein using bicinchoninic acid. Anal. Biochem. 150, 76-85 (1985)).

Binding experiments: Crude D2/D3 cell membranes were incubated with 0.03 nM [¹²⁵I] Iodosulpride (˜2000 Ci/mmol; Amersham, U. K., and the test compound in a buffer containing 50 mM Trizma pre-set crystals (pH 7.4 @ 37° C.), 120 mM NaCl, 5 mM KCl, 2 mM CaCl₂, 1 mM MgCl₂, 0.3% (w/v) bovine serum albumin. The total volume is 0.2 ml and incubated in a water bath at 37° C. for 40 minutes. Following incubation, samples were filtered onto GF/B Unifilters using a Can berra Packard Filtermate, and washed four times with ice-cold 50 mM Trizma pre-set crystals (pH 7.4 @ 37° C.). The radioactivity on the filters was measured using a Can berra Packard Topcount Scintillation counter. Non-specific binding was defined with 10 μM SKF-102161 (YM-09151). For competition curves, 10 serial log concentrations of competing cold drug were used (Dilution range: 10 μM-10 pM). Competition curves were analysed using Inflexion, an iterative curve fitting programme in Excel. Results were expressed as pKᵢ values where
pKᵢ=−log10[Ki].

The exemplified compounds have pKᵢ values within the range of 7.1-9.4 at the dopamine D₃ receptor.

The exemplified compounds have pKᵢ values within the range of 6.0-8.8 at the dopamine D₂ receptor.

Binding Experiments on Cloned 5-HT₆ Receptors

Compounds can be tested following the procedures outlined in WO 98/27081.

The exemplified compounds have pKᵢ values within the range of 6.4-9.0 at the serotonin 5-HT₆ receptor.

Binding Experiments on Cloned 5-HT_{2A} and 5-HT_{2C} receptors

Compounds can be tested following the procedures outlined in WO 94/04533.

The exemplified compounds have pKᵢ values within the range of 6.3-9.2 at the serotonin 5-HT_{2A} and 5-HT_{2C} receptors.

The invention is further illustrated by the following non-limiting examples:

Description 1

### 1-(7-Amino-1,2,4,5-tetrahydro-3-benzazepin-3-yl)-2,2,2-trifluoro-ethanone (D1)

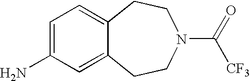

The title compound was prepared from 7-nitro-1,2,4,5-tetrahydro-3-benzazepine by reaction with trifluoroacetic anhydride followed by hydrogenation, using a procedure similar to that described in WO 9914197. MH⁺ 259.

Description 2

### 1-(7-Amino-3,4-dihydro-1H-isoquinolin-2-yl)-2,2,2-trifluoro-ethanone (D2)

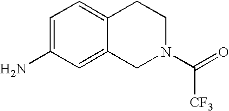

The title compound was prepared using a similar methodology to that described in WO 9914197. MH⁺ 245.

Description 3

### 7-Amino-8-methoxy-1,2,4,5-tetrahydro-3-benzazepine-3-carboxylic Acid tert-butyl Ester (D3)

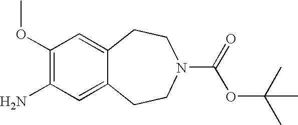 a) 7-Methoxy-1,2,4,5-tetrahydro-3-benzazepine-3-carboxylic Acid Tert-butyl Ester

To a solution of 7-hydroxy-1,2,4,5-tetrahydro-3-benzazepine-3-arboxylic acid tert-butyl ester (5 g, 19 mmol) in dimethylformamide (50 mL) was added potassium carbonate (3.4 g, 25 mmol) and methyl iodide (3.25 mL, 60 mmol). The mixture was heated to 30° C. for 12 h. The solvent was evaporated and the residue partitioned between dichloromethane (100 mL) and water (100 mL). The organic layer was separated and evaporated to give the crude product as a colourless oil (5.3 g, 100%).

b) 7-Methoxy-8-nitro-1,2,4,5-tetrahydro-3-benzazepine-3-carboxylic Acid Tert-butyl Ester

To a mixture of 7-methoxy-1,2,4,5-tetrahydro-3-benzazepine-3-carboxylic acid tert-butyl ester (5.3 g, 19 mmol) in glacial acetic acid (100 mL) and acetic anhydride (10 mL) at 0° C. was added a mixture of nitric acid (70% aqueous, 5 g, 55 mmol) dropwise in glacial acetic acid (100 mL) and acetic anhydride (10 mL) maintaining the temperature below 5° C. The mixture was stirred at room temperature for 2 h and then poured into ice/water (500 ml). The aqueous was extracted with dichloromethane (2×200 mL) and the combined organic portions were neutralised with saturated sodium bicarbonate solution. The dichloromethane layer was evaporated and the residue chromatographed on silica gel (eluent: hexane/dichloromethane (1:1) to dichloromethane) to give the product as a colourless solid (1.5 g, 25%).

c) 7-Amino-8-methoxy-1,2,4,5-tetrahydro-3-benzazepine-3-carboxylic Acid Tert-butyl Ester

To a solution of 7-methoxy-8-nitro-1,2,4,5-tetrahydro-3-benzazepine-3-carboxylic acid tert-butyl ester (1.5 g, 4.7 mmol) in ethanol (80 mL) was added palladium on charcoal (10%, 0.5 g). The mixture was stirred under an atmosphere of hydrogen for 2 h and then filtered. The solvent was evaporated to give the title compound as a colourless solid (1.35 g, 100%).

Mass spectrum AP⁺: Found 193 ([M-Boc]⁺). C₁₆H₂₄N₂O₃ requires 292. 1H NMR (CDCl₃) δ 1.48 (9H, s), 2.76 (4H, m), 3.51 (4H, m), 3.65 (2H, s), 3.82 (3H, s), 6.50 (1H, m), 6.56 (1H, m).

Description 4

### 5-Amino-1,3-dihydro-isoindole-2-carboxylic Acid Tert-butyl Ester (D4)

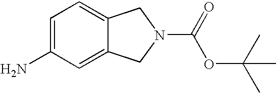

The title compound was prepared from 5-nitro-isoindoline by reaction with di-t-butyl dicarbonate followed by hydrogenation, using a similar procedure to that described in WO 9914197. ¹H NMR: 8 CDCl₃ 1.52 (9H, s), 4.74 (2H, s), 4.77 (2H, s), 7.4 (1H, m), 8.2 (2H, m).

### EXAMPLE 1

### 4-Butyl-N-(2,3,4,5-tetrahydro-1H-3-benzazepin-7-yl)-benzenesulfonamide (E1)

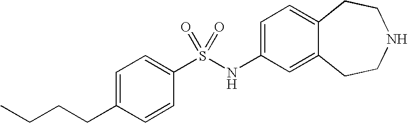 a) 4-Butyl-N-[3-(2,2,2-trifuoro-ethanoyl)-2,3,4,5-tetrahydro-1H-3-benzazepin-7-yl]-benzenesulfonamide

The amine D1 (0.5 g, 1.9 mmol) was dissolved in pyridine (8 mL) and cooled to 0° C. To this stirred solution was added dropwise a solution of 4-n-butylphenylsulfonyl chloride (0.89 g, 3.8 mmol), and the resultant mixture stirred at room temperature for 18 h. The reaction mixture was then poured onto brine and extracted with dichloromethane. The combined organic layers were washed with citric acid solution followed by brine, then dried and evaporated. Chromatography of the crude product on silica eluting with 30% ethyl acetate/hexane afforded the product (0.7 g) MH⁺ 398.

b) 4-Butyl-N-(2,3,4,5-tetrahydro-1H-3-benzazepin-7-yl)-benzenesulfonamide

The product from a) was dissolved in 2M ammonia in methanol (30 mL) and water (6 mL) added to the stirred solution. Stirring was continued for 18 h, then the solution evaporated to dryness. Application of the crude product to an SCX ion exchange cartridge, followed by elution with methanol followed by 1% ammonia in methanol afforded the title compound (0.44 g). MH⁺ 359. ¹H NMR: 8 CDCl₃ 0.91 (3H, t), 1.29 (2H, m), 1.57 (2H, m), 2.63 (2H, t), 2.82 (4H, m), 2.90 (4H, m), 6.78 (2H, m), 6.94 (1H, d), 7.25 (2H, d), 7.65 (2H, d).

### EXAMPLE 2

### 4-Butyl-N-(3-methyl-2,3,4,5-tetrahydro-1H-3-benzazepin-7-ylybenzenesulfonamide (E2)

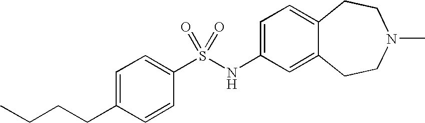

A solution of E1 (140 mg) in dichloroethane (20 mL) was treated with formalin (0.3 mL) followed by sodium triacetoxyborohydride (350 mg). The mixture was stirred for 18 h, then added to sodium bicarbonate solution and extracted with dichloromethane. The combined organic extracts were washed with brine, dried and evaporated to afford the crude product. Chromatography on silica, eluting with 2% methanol in dichloromethane containing 0.5% aqueous ammonia, afforded the title compound (47 mg). MH⁺ 373. ¹H NMR: 5 CDCl₃ 0.91 (3H, t), 1.32 (2H, m), 1.57 (2H, m), 2.34 (3H, s), 2.50 (4H, m), 2.63 (2H, t), 2.83 (4H, m), 6.76 (2H, m), 6.94 (1H, d), 7.24 (2H, d), 7.64 (2H, d).

### EXAMPLE 3

### 4-Butyl-N-methyl-N-(2,3,4,5-tetrahydro-1H-3-benzazepin-7-yl)benzenesulfonamide hydrochloride (E3)

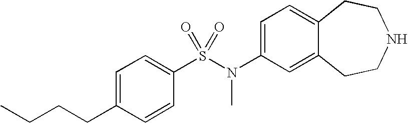 a) 4-Butyl-N-methyl-N-[3-(2,2,2-trifluoro-ethanoyl)-2,3,4,5-tetrahydro-1H-3-benzazepin-7-yl]-benzenesulfonamide

The trifluoroacetamide E1a (205 mg) was dissolved in dry tetrahydrofuran (7 mL) containing triphenylphosphine (150 mg) and dry methanol (150 mg). To this stirred solution was added di-isopropylazodicarboxylate (113 mg) and the mixture stirred at room temperature for 18 h. The solvent was then evaporated and the residue chromatographed on silica using 12% ethyl acetate/hexane as eluant to afford the product (200 mg). MH⁺ 468.

b) 4-Butyl-N-methyl-N-(2,3,4,5-tetrahydro-1H-3-benzazepin-7-yl)-benzenesulfonamide Hydrochloride

Deprotection of the product from a) using a procedure similar to that in E1b afforded the title compound (180 mg), which was isolated as the hydrochloride salt. MH⁺ 373. ¹H NMR: δ CDCl₃ (free base) 0.93 (3H, t), 1.35 (2H, m), 1.62 (2H, m), 2.67 (2H, t), 2.88 (8H, m), 3.13 (3H, s), 6.76 (1H, m), 6.82 (1H, s), 6.99 (1H, d), 7.23 (2H, d), 7.47 (2H, d), 8.01 (1H, s).

### EXAMPLE 4

### 4-Butyl-N-methyl-N-3-methyl-2,3,4,5-tetrahydro-1H-3-benzazepin-7-yl)-benzenesulfonamide Hydrochloride (E4)

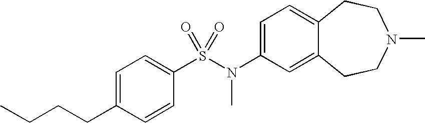

The title compound was prepared from Example 3 using a procedure similar to that for Example 2, and the product isolated as the hydrochloride salt. MH⁺ 387. ¹H NMR: δ CDCl₃ 0.93 (3H, t), 1.36 (2H, m), 1.60 (2H, m), 2.37 (3H, s), 2.56 (4H, b s), 2.67 (2H, t), 2.86 (4H, b s), 3.13 (3H, s), 6.78 (1H, m), 6.83 (1H, s), 6.99 (1H, d), 7.25 (2H, d), 7.46 (2H, d).

### EXAMPLE 5

### 4-Butyl-N-(8-methoxy-2,3,4,5-tetrahydro-1H-3-benzazepin-7-yl)-benzenesulfonamide Hydrochloride (E5)

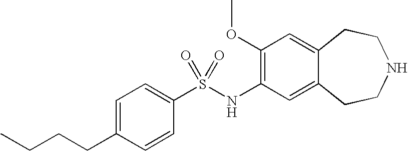 a) 7-(4-Butyl-benzenesulfonylamino)-8-methoxy-1,2,4,5-tetrahydro-3-benzazepine-3-carboxylic Acid Tert Butyl Ester

The BOC protected intermediate was prepared from D3 and 4-n-butylphenylsulfonyl chloride using a procedure analogous to that for Example 1a. MH⁺ 489.

b) 4-Butyl-N-(8-methoxy-2,3,4,5-tetrahydro-1H-3-benzazepin-7-yl)benzenesulfonamide Hydrochloride

The title compound was prepared from a) by treatment with a solution of ethanolic hydrogen chloride, followed by the addition of ether to precipitate the product. MH⁺ 389. ¹H NMR: δ DMSO 0.88 (3H, t, =7.3 Hz), 1.27 (2H, m), 1.52 (2H, m), 2.62 (2H, t, J=7.6 Hz), 2.99 (4H, m), 3.09 (4H, m), 3.32 (3H, s), 6.78 (1H, s), 7.04 (1H, s), 7.33 (2H, d, 3=8.3 Hz), 7.59 (2H, d, J=8.3 Hz), 9.18 (3H, broad m).

### EXAMPLE 6

### 4-Butyl-N-(3-methyl-8-methoxy-2,3,4,5-tetrahydro-H-3-benzazepin-7-yl)-benzenesulfonamide Hydrochloride (E6)

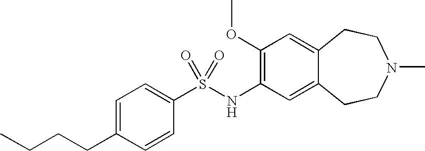

The title compound was prepared from Example 5 using a procedure similar to that for Example 2, and the product isolated as the hydrochloride salt. MH⁺ 403. ¹H NMR: 8 CDCl₃ 0.89 (3H, m), 1.23-1.38 (2H, m), 1.5-1.62 (2H, m), 2.35 (3H, s), 2.47-2.53 (4H, m), 2.58-2.64 (2H, m), 2.79-2.87 (4H, m), 3.54 (3H, s), 6.46 (1H, s), 6.70-6.95 (1H, br, s), 7.16-7.20 (2H, m), 7.25 (1H, s), 7.59-7.65 (2H, m).

Examples 10-76 and 82 were prepared using analogous procedures to Examples 1-6 using the appropriate starting materials, with the products being isolated as either the free bases or hydrochloride salts. All ¹H NMR are consistent with the structures shown.

### EXAMPLE 7

### 4-Butyl-N-(1,2,3,4-tetrahydro-isoquinolin-7-yl)-benzenesulfonamide (E7)

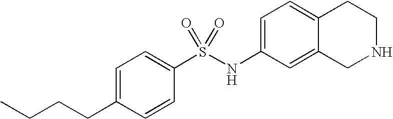

The title compound was prepared from the aniline D2 and 4-n-butylphenylsulfonyl chloride using a procedure similar to Example 1. MH⁺ 345. ¹H NMR: δ CDCl₃ 0.91 (3H, t), 1.33 (2H, m), 1.57 (2H, m), 2.63 (2H, t), 2.71 (2H, t), 3.08 (2H, t), 3.89 (2H, s), 6.72 (1H, s), 6.79 (1H, m), 6.93 (1H, d), 7.23 (2H, d), 7.63 (2H, d).

Examples 38-54 and 77-81 were prepared using analogous procedures to Examples 1-7 using the appropriate starting materials, with the products being isolated as either the free bases or hydrochloride salts. All ¹H NMR are consistent with the structures shown.

### EXAMPLE 8

### 4-Butyl-N-(2,3-dihydro-1H-isoindol-5-yl)benzenesulfonamide Hydrochloride (E8)

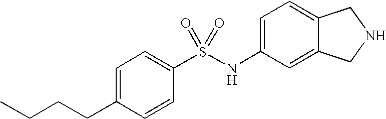

The title compound was prepared from D4 and 4-n-butylphenylsulfonyl chloride using a procedure to that for Examples 1a and 5b. MH⁺ 331. ¹H NMR: δ DMSO 0.87 (3H, m), 1.2 (2H, m), 1.5 (2H, m), 2.62 (2H, m), 4.4 (4H, m), 7.07 (1H, d), 7.16 (1H, s), 7.24 (2H, d), 7.35 (2H, m), 7.68 (2H, d), 9.8 (2H, m), 10.46 (1H, m).

### EXAMPLE 9

### 4-Butyl-N-(2-methyl-2,3-dihydro-1H-isoindol-5-yl)benzenesulfonamide (E9)

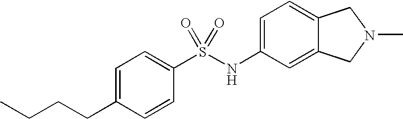

The title compound was prepared from E8 using a procedure similar to that for E2. MH⁺ 345. ¹H NMR: 3 DMSO 0.86 (3H, m), 1.24 (2H, m), 1.54 (2H, m), 2.49 (2H, s), 2.61 (2H, m), 3.68 (4H, s), 6.88 (1H, d), 6.93 (1H, s), 7.05 (H, d), 7.35 (2H, d), 7.64 (2H, d), 10.1 (1H, m).

All of the compounds listed below in Table 1 relate to compounds of formula (IF):

TABLE 1
(IF)

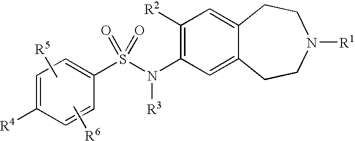

| Example | R¹ | R² | R³ | R⁴ | R⁵ | R⁶ | MH⁺ |
|---|---|---|---|---|---|---|---|
| 1 | H | H | H | n-butyl | H | H | 359 |
| 2 | Me | H | H | n-butyl | H | H | 373 |
| 3 | H | H | Me | n-butyl | H | H | 373 |
| 4 | Me | H | Me | n-butyl | H | H | 387 |
| 5 | H | OMe | H | n-butyl | H | H | 389 |
| 6 | Me | OMe | H | n-butyl | H | H | 403 |
| 10 | H | H | H | n-propyl | H | H | 345 |
| 11 | Me | H | H | n-propyl | H | H | 359 |
| 12 | H | H | H | i-propyl | H | H | 345 |
| 13 | Me | H | H | i-propyl | H | H | 359 |
| 14 | H | H | H | 1,1-diMepropyl | H | H | 373 |
| 15 | Me | H | H | 1,1-diMepropyl | H | H | 387 |
| 16 | Me | H | H | t-butyl | H | H | 373 |
| 17 | H | H | H | n-pentyl | H | H | 373 |
| 18 | Me | H | H | n-pentyl | H | H | 387 |
| 19 | Me | H | H | cyclohexyl | H | H | 399 |
| 20 | Me | H | H | I | H | H | 443 |
| 21 | Me | H | H | CF₃ | H | H | 385 |
| 22 | Me | H | H | OCF₃ | H | H | 401 |
| 23 | H | H | H | O-propyl | H | H | 361 |
| 24 | Me | H | H | O-propyl | H | H | 375 |
| 25 | H | H | H | O-butyl | H | H | 375 |
| 26 | Me | H | H | O-butyl | H | H | 389 |
| 27 | Et | H | H | n-butyl | H | H | 387 |
| 28 | n-Pr | H | H | n-butyl | H | H | 401 |
| 29 | i-Pr | H | H | n-butyl | H | H | 401 |
| 30 | H | H | Et | n-butyl | H | H | 387 |
| 31 | Me | H | Et | n-butyl | H | H | 401 |
| 32 | H | H | n-Pr | n-butyl | H | H | 401 |
| 33 | Me | H | n-Pr | n-butyl | H | H | 415 |
| 34 | H | H | i-Pr | n-butyl | H | H | 401 |
| 35 | Me | H | i-Pr | n-butyl | H | H | 415 |
| 36 | H | Br | H | n-butyl | H | H | 438 |
| 37 | H | EtO | H | n-butyl | H | H | 403 |
| 55 | Me | EtO | H | n-butyl | H | H | 417 |
| 56 | H | MeO | H | n-propyl | H | H | 375 |
| 57 | H | MeO | H | i-propyl | H | H | 375 |
| 58 | H | MeO | H | 1,1-diMepropyl | H | H | 403 |
| 59 | H | MeO | H | n-pentyl | H | H | 403 |
| 60 | H | MeO | H | c-hexyl | H | H | 415 |
| 61 | H | MeO | H | OCF₃ | H | H | 417 |
| 62 | Me | MeO | H | OCF₃ | H | H | 431 |
| 63 | Me | MeO | H | O-propyl | H | H | 391 |
| 64 | Me | MeO | H | O-butyl | H | H | 405 |
| 65 | H | i-PrO | H | n-butyl | H | H | 417 |
| 66 | Me | i-PrO | H | n-butyl | H | H | 431 |
| 67 | H | MeO | Me | n-butyl | H | H | 403 |
| 68 | Me | MeO | Me | n-butyl | H | H | 417 |
| 69 | H | MeO | i-Pr | n-butyl | H | H | 431 |
| 70 | Me | MeO | i-Pr | n-butyl | H | H | 445 |
| 71 | H | Et | H | n-butyl | H | H | 387 |
| 72 | Me | Et | H | n-butyl | H | H | 401 |
| 73 | Me | (2-thienyl) | H | n-butyl | H | H | 455 |
| 74 | H | (2-thienyl) | H | n-butyl | H | H | 441 |
| 75 | Me | (2-furyl) | H | n-butyl | H | H | 425 |
| 76 | Me | Br | H | n-butyl | H | H | 452 |
| 82 | Me | MeO | H | cyclohexyl | H | H | 429 |

All of the compounds listed below in Table 2 relate to compounds of formula (IC):

TABLE 2
(IC)

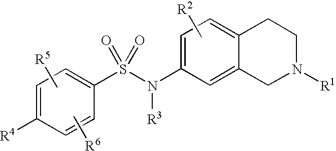

| Example | R¹ | R² | R3 | R⁴ | R⁵ | R⁶ | MH⁺ |
|---|---|---|---|---|---|---|---|
| 7 | H | H | H | n-butyl | H | H | 345 |
| 38 | Me | H | H | n-butyl | H | H | 359 |
| 39 | Me | H | H | n-propyl | H | H | 345 |
| 40 | Me | H | H | i-propyl | H | H | 345 |
| 41 | H | H | H | n-pentyl | H | H | 359 |
| 42 | Me | H | H | n-pentyl | H | H | 373 |
| 43 | H | H | H | cyclohexyl | H | H | 371 |
| 44 | Me | H | H | cyclohexyl | H | H | 385 |
| 45 | H | H | H | O-n-butyl | H | H | 361 |
| 46 | Me | H | H | O-n-butyl | H | H | 375 |
| 47 | Et | H | H | n-butyl | H | H | 373 |
| 48 | n-Pr | H | H | n-butyl | H | H | 387 |
| 49 | i-Pr | H | H | n-butyl | H | H | 387 |
| 50 | H | H | Me | n-butyl | H | H | 359 |
| 51 | Me | H | Me | n-butyl | H | H | 373 |
| 52 | H | H | Et | n-butyl | H | H | 373 |
| 53 | Me | H | Et | n-butyl | H | H | 387 |
| 54 | Me | H | i-Pr | n-butyl | H | H | 401 |
| 77 | H | 6-MeO | H | i-propyl | H | H | 361 |
| 78 | H | 6-MeO | H | n-butyl | H | H | 375 |
| 79 | H | 6-MeO | H | c-hexyl | H | H | 401 |
| 80 | H | 6-MeO | H | OCF₃ | H | H | 403 |
| 81 | H | 6-MeO | H | O-n-Pr | H | H | 377 |

All of the compounds listed below in Table 3 relate to compounds of formula (IB):

TABLE 3
(IB)

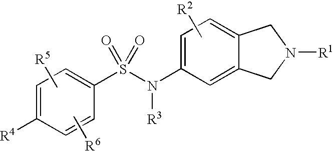

| Example | R¹ | R² | R³ | R⁴ | R⁵ | R⁶ | MH⁺ |
|---|---|---|---|---|---|---|---|
| 8 | H | H | H | n-butyl | H | H | 331 |
| 9 | Me | H | H | n-butyl | H | H | 345 |

1. A compound of formula (I) 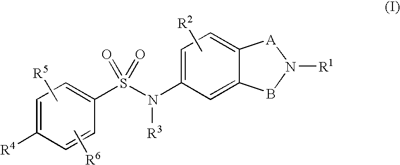
wherein
A and B represent the groups CH₂)ₘ— and —(CH₂)ₙ— respectively;
R¹ represents hydrogen or C₁₋₆alkyl;
R² represents hydrogen, halogen, hydroxy, cyano, nitro, hydroxyC₁₋₆alkyl, trifluoromethyl, trifluoromethoxy, C₁₋₆alkyl, C₁₋₆alkoxy, —(CH₂)ₚC₃₋₆cycloalkyl, —(CH₂)ₚC₃₋₆cycloalkyloxy, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl or a fused bicyclic heterocyclic ring system;
R³ represents hydrogen or C₁₋₆alkyl;
R⁴ represents halogen, trifluoromethyl, trifluoromethoxy, C₁₋₆alkyl, C₁₋₆alkoxy, —(CH₂)ₚC₃₋₆cyclo alkyl or —(CH₂)ₚC₃₋₆cycloalkyloxy;
R⁵ and R⁶ each independently represent hydrogen, halogen, hydroxy, cyano, nitro, hydroxyC₁₋₆alkyl, trifluoromethyl, trifluoromethoxy, C₁₋₆alkyl, C₁₋₆alkoxy, —(CH₂)ₚC₃₋₆cycloalkyl, —(CH₂)ₚC₃₋₆cycloalkyloxy, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl or a fused bicyclic heterocyclic ring system;
R⁷ and R⁸ each independently represent hydrogen or C₁₋₆alkyl;
m and n independently represent an integer selected from 1 and 2;
p independently represents an integer selected from 0, 1, 2 and 3;
or a pharmaceutically acceptable salt or solvate thereof,
with the proviso that the compounds 4-methyl-N-(1,2,3,4-tetrahydroisoquinolin-6-yl)-benzenesulfonamide, 7-(4-chlorophenyl)sulfonamido-1,2,3,4-tetrahydroisoquinoline hydrochloride and N-(2-ethyl-5-isoindolinyl)-p-toluenesulfonamide are excluded.

2. A compound of formula (I) which is
4-Butyl-N-(2,3,4,5-tetrahydro-1H-3-benzazepine-7-yl)-benzenesulfonamide;
4-Butyl-N-(3-methyl-2,3,4,5-tetrahydro-1H-3-benzazepine-7-yl)-benzenesulfonamide;
4-Butyl-N-methyl-N-(2,3,4,5-tetrahydro-1H-3-benzazepine-7-yl)-benzenesulfonamide hydrochloride;
4-Butyl-N-methyl-N-(3-methyl-2,3,4,5-tetrahydro-1H-3-benzazepine-7-yl)-benzenesulfonamide hydrochloride;
4-Butyl-N-(8-methoxy-2,3,4,5-tetrahydro-1H-3-benzazepine-7-yl)-benzenesulfonamide hydrochloride;
4-Butyl-N-(3-methyl-8-methoxy-2,3,4,5-tetrahydro-1H-3-benzazepine-7-yl)-benzenesulfonamide hydrochloride;
4-Butyl-N-(1,2,3,4-tetrahydro-isoquinolin-7-yl)-benzenesulfonamide;
4-Butyl-N-(2,3-dihydro-1H-isoindol-5-yl)-benzenesulfonamide hydrochloride; and
4-Butyl-N-(2-methyl-2,3-dihydro-1H-isoindol-5-yl)-benzenesulfonamide.

3. A pharmaceutical composition comprising a compound of formula (I) as claimed in claim 1 or a pharmaceutically acceptable salt thereof and a pharmaceutically acceptable carrier therefor.

4. A compound of formula (I) or a pharmaceutically acceptable salt or solvate thereof as claimed in claim 1, for use in therapy.

5. A compound of formula (I) or a pharmaceutically acceptable salt or solvate thereof as claimed in claim 1 for use in a condition which requires modulation of a dopamine receptor.

6. A compound of formula (I) or a pharmaceutically acceptable salt or solvate thereof according to claim 5 wherein the condition is selected from psychotic disorders, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive compulsive disorders, amnesia, aggression, autism, vertigo, dementia, circadian rhythm disorders and gastric motility disorders.

7. (canceled)

8. (canceled)

9. A method of treating a condition which requires modulation of a dopamine receptor, which comprises administering to a mammal in need thereof an effective amount of a compound of formula (I) or a pharmaceutically acceptable salt or solvate thereof as claimed in claim 1.

10. A method of treating a condition according to claim 9 wherein the condition is selected from psychotic disorders, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.
